(19) United States
(12) Patent Application Publication
(10) Pub. No.: US 2009/0163765 A1
(43) Pub. Date: Jun. 25, 2009

(54) Catheter for artificial insemination in animals

(76) Inventor: Sheng-Jui Chen, Pyng-Jenn City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/003,256

(22) Filed: Dec. 21, 2007

(51) Int. Cl. A61B 17/43

(52) U.S. Cl. 600/35

(57) ABSTRACT

The invention discloses an improved catheter for artificial insemination in animals, comprising primarily a simple improvement of the nozzle mounted at the front end of catheter. The nozzle is predefined on its peripheral surface with pores, channels, or indented texts or patterns of different shapes, which can be used to hold reagents, liquid medicine, ointment or lubricant for animals. As such, when the operator inserts the nozzle into the vagina of female animal body, the procedure achieves not only artificial insemination, but also other purposes, such as testing, experiment and treatment, thereby augmenting the usage and enhancing the efficiency of artificial insemination catheter.

### BACKGROUND OF THE INVENTION

1) Field of the Invention

The present invention relates to an improved catheter for artificial insemination in animals, more particularly an improved catheter for artificial insemination in animals that is easy and convenient to use, and provides multiple functions including artificial insemination, and in addition, testing, experiment and treatment, thereby augmenting the usage of catheter and enhancing its efficiency. The invention is practical, ideal and inventive.

2) Description of the Prior Art

The assembly of a conventional artificial insemination device for animals as shown in FIG. 1 and FIG. 2 typically comprises a catheter tube 10 and a nozzle 20 installed at the front end of catheter tube 10. The nozzle 20 is typically made of flexible material (e.g. foam or synthetic rubber) so it can be pushed into the vagina and uterus of female animal body 1. The nozzle 20 is generally conical (as shown in FIG. 1) or spiral (as shown in FIG. 2) in shape. The peripheral surface of the conical-shaped nozzle 20 is round and smooth so as to reduce friction and not injure the superficial tissues of the vagina and uterus of female animal body when it is inserted. At the middle section of the peripheral surface there is provided a concave sensor groove 201 of proper depth. When the nozzle 20 enters the uterus along the vagina, the sensor groove 201 would enable the nozzle to produce different tactile pressure when it senses the contraction of the uterus. The operator with his hand holding the catheter tube could sense the pressure and realize that the nozzle 20 has entered the uterus of the animal. The spiral-shaped nozzle 20 has on its peripheral surface spiral grooves 202 and enters the vagina and uterus of the female animal body through the rotation of the catheter tube 10. The spiral grooves 202 also enable the nozzle 20 to produce distinctly different tactile pressure in the presence of uterine contraction and thereby inform the operator of the location of nozzle in animal body.

The aforementioned artificial insemination assembly and operation undeniably achieves the intended effect and is practically the sole approach adopted in the industry at the present time. However operators still find some drawbacks after prolonged use. For instance, when performing artificial insemination in animals, operators would first coat lubricant around the nozzle to reduce the friction between nozzle and the vagina and uterus of animal. However in practice, regardless whether the nozzle enters the animal vagina and uterus by push-in or turning, its peripheral surface is invariably in full contact with the vagina such that the coated lubricant would have been completely rubbed off before the nozzle approaches the uterus, hence cutting back the expected effect of lubrication. Moreover, the assembly of catheter tube and nozzle, be it in conical or spiral shape, is used simply for the purpose of artificial insemination, which constitutes a waste in implementation.

In view of the drawbacks just mentioned, the inventor combines practical and theoretical knowledge and research to improve the conventional design and comes up with the present invention.

### SUMMARY OF THE INVENTION

The primary object of the present invention is to provide an improved catheter for artificial insemination in animals, characterized in which the nozzle attached to the catheter tube is predefined on its peripheral surface with pores, channels, or indented texts or patterns of different shapes, which can be used to hold reagents, liquid medicine, ointment or lubricant for animals. When the nozzle is mounted on a catheter tube and inserted into the vagina and uterus of female animal body, the procedure achieves not only artificial insemination, but also other purposes, such as testing, experiment and treatment as needed in view of the physical or physiological conditions of the animal, thereby augmenting the usage and enhancing the efficiency of artificial insemination catheter.

Another object of the present invention is to provide an improved catheter for artificial insemination in animals, characterized in which the nozzle attached to the catheter tube is predefined on its peripheral surface with pores, channels, or indented texts or patterns of different shapes, which achieves the recognition and aesthetic effects. In addition, such nozzle uses less material, hence rendering its production more economical.

### BRIEF DESCRIPTION OF THE DRAWINGS

FIG. 1 is a perspective view of a conventional catheter for artificial insemination in animals.

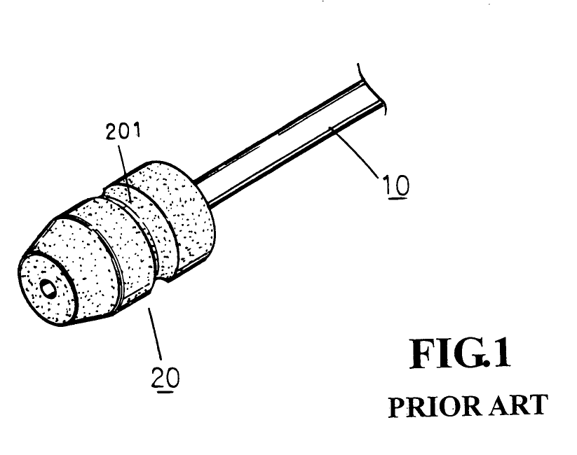

FIG. 2 is a plane view of a conventional catheter for artificial insemination in animals.

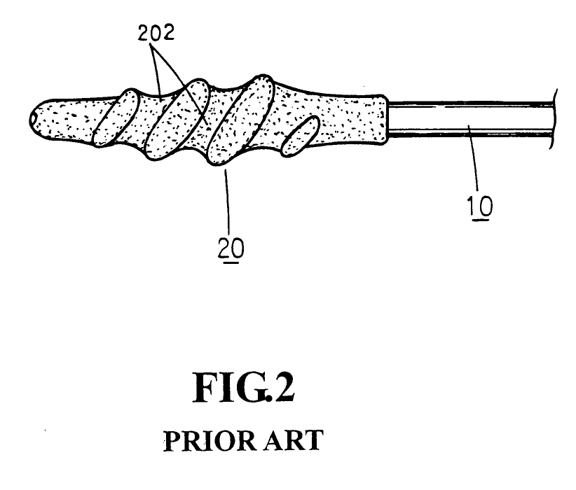

FIG. 3 is a perspective view (1) of a catheter for artificial insemination in animals according to embodiment of the invention.

FIG. 4 is a plane view of FIG. 3.

FIG. 5 is a perspective view (2) of a catheter for artificial insemination in animals according to embodiment of the invention.

### DETAILED DESCRIPTION OF THE INVENTION

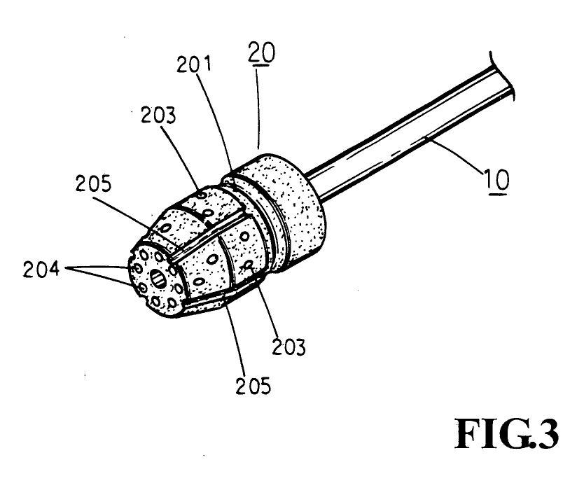

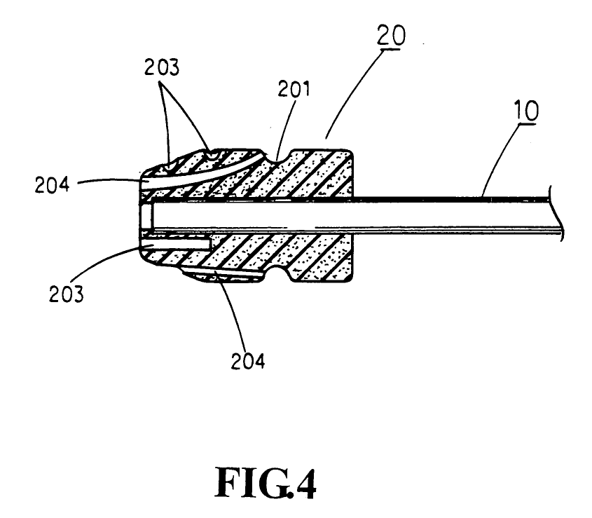

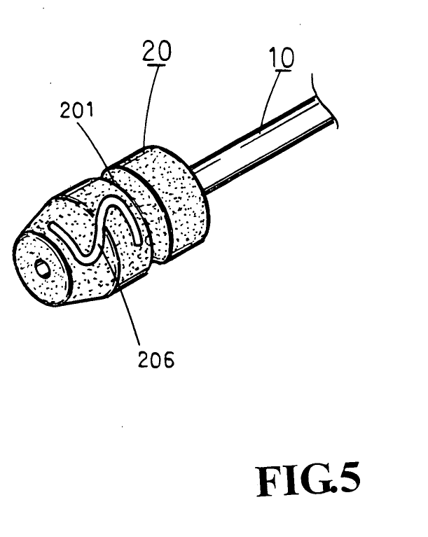

Referring to FIGS. 3, 4, and 5, the improved catheter for artificial insemination in animals according to the invention comprises a simple improvement of nozzle 20 mounted at the front end of a catheter 10. That is, the nozzle 20 is predefined on its peripheral surface with a plurality of concave pores 203, through holes 204, channels 205, or indented texts or patterns 206 of different shapes such that the nozzle 20 can hold lubricant, liquid medicine or ointment by means of coating, soaking or spraying before it enters the vagina and uterus of the female animal body.

When performing artificial insemination in animals, the operator can coat the lubricant by various means around the nozzle 20, while the plurality of pores, holes, channels, indented texts or patterns thereon can hold the lubricant. Subsequently as the nozzle 20 is pushed into the vagina and uterus of the female animal body by the catheter 10, the lubricant in the pores, holes, channels, and indented texts and patterns will be squeezed out as the flexible nozzle is pushed and squeezed in the procedure. As such, the peripheral surface of the nozzle 20 is always adhered with lubricant, thereby achieving the original purpose and expected effect of lubricating the surface of nozzle 20 in artificial insemination.

In addition to holding lubricant as described above, the pores, holes, channels, or indented texts or patterns predefined on the peripheral surface of nozzle 20 can also hold reagents, liquid medicine or ointment used in the testing, experiment or treatment of animals. For example, if reagent is placed in the pores, channels, indented texts or patterns on the surface of nozzle 20, when the nozzle 20 enters the vagina or uterus of the female animal body, the spilled over reagent can be mixed with the secretions of the animal to learn whether the animal is in heat, has inflammation or other conditions so operator would have a better grasp of the animal's condition. If medicine is placed in the pores, channels, indented texts or patterns on the surface of nozzle 20, when the nozzle 20 enters the vagina or uterus of the female animal body, the spilled over medicine can provide treatment effect or promote the uterine contraction to enhance the efficiency of artificial insemination and the chance of conception.

As described above, the pores, channels, indented texts or patterns on the surface of nozzle 20 predefined around the surface of nozzle 20 can be used to hold lubricant, reagent, medicine or ointment, thereby allowing the artificial insemination catheter for animals to provide multiple functions. Such nozzle design can offer advertising effect. In addition, with the pores, channels, indented texts or patterns disposed on nozzle 20, it helps save the use of material in the molding of nozzle, thereby rendering the nozzle production more economical.

To prevent semen injected into the uterus of female animal body to outflow from behind the nozzle 20, those predefined pores, channels, or indented texts or patterns are designed in such a way that they do not communicate with the backend surface of nozzle 20. If a sensor groove 201 is arranged on the peripheral surface of nozzle 20, those pores, channels, or indented texts or patterns preferably communicate with the sensor groove 201. In particular, when the nozzle 20 enters the uterus, the uterine contraction would cause convection of the reagent or liquid medicine held in the pores, channels, or indented texts or patterns that communicate with the sensor groove 201, thereby accelerating the release of reagent or liquid medicine.

To sum up, the improved catheter for artificial insemination in animals provides better functions than prior art without requiring complex or cumbersome structure or assembly. Its operation takes full consideration of practicality in implementation and its applications are not seen in similar products currently available. Thus the present invention meets the criteria for novelty and inventive step.

1. An improved catheter for artificial insemination in animals, comprising a catheter and a flexible nozzle mounted at the front end of catheter, characterized in which:
the nozzle being arranged on its peripheral surface with a plurality of concave pores, through holes, channels, or indented texts or patterns of different shapes for holding reagents, liquid medicine, ointment or lubricant for animals therein.

2. The improved catheter for artificial insemination in animals according to claim 1, wherein the through holes, channels and indented texts or patterns do not communicate with the backend surface of nozzle.
